(19) United States
(12) Patent Application Publication
(10) Pub. No.: US 2024/0269148 A1
(43) Pub. Date: Aug. 15, 2024

(54) Enhancement of Anti-Angiogenic Cancer Immunotherapy by Abortogenic Agents

(71) Applicant: Therapeutic Solutions International, Inc., Oceanside, CA (US)

(72) Inventors: Thomas E. Ichim, Oceanside, CA (US); Famela Ramos, Oceanside, CA (US); James Veltmeyer, OCEANSIDE, CA (US); Timothy G. Dixon, Oceanside, CA (US)

(73) Assignee: Therapeutic Solutions International, Inc., Oceanside, CA (US)

(21) Appl. No.: 18/443,160

(22) Filed: Feb. 15, 2024

Related U.S. Application Data
(60) Provisional application No. 63/445,872, filed on Feb. 15, 2023.

(51) Int. Cl. A61K 31/567; A61K 35/44; A61K 45/06; A61P 35/00

(52) U.S. Cl. CPC A61K 31/567; A61K 35/44; A61K 45/06; A61P 35/00

(57) ABSTRACT

Parallels between pregnancy and cancer have been historically made, however, the ability to leverage abortogenic immunity against neoplasia has not been widely examined. The current invention provides means of suppressing tumor associated immune inhibition through administration of progesterone and/or glucocorticoid receptor antagonists such as RU-486. In one embodiment the invention provides the concurrent utilization RI-486 and anti-angiogenic immunotherapy. In another embodiment, abortogenic inhibitors of immunity such as indolamine 2,3 dioxygenase are administered together with RU-486 and/or anti-angiogenic immunotherapy. Various antiangiogenic agents can be utilized in the practice of the invention including the ValloVax immunotherapy and/or the StemVacs-V therapy.

### CROSS-REFERENCE TO RELATED APPLICATIONS

The present application claims benefit of U.S. Provisional Patent Application Ser. No. 63/445,872, filed on Feb. 15, 2023, entitled “Enhancement of Anti-Angiogenic Cancer Immunotherapy by Abortogenic Agents”, the contents of which are incorporated herein by reference in its entirety.

### FIELD OF THE INVENTION

The teachings herein relate to the treatment of cancer through administration of one or more abortogenic agents combined with one or more immunotherapies.

### BACKGROUND OF THE INVENTION

The use of the immune system to kill cancer has been proposed for more than a century. However only until recently has it been possible to obtain therapeutic results with some level of reproducibility. This is because previous attempts to induce anticancer immunity relied on molecularly ill defined preparations such as Coley's vaccine, which possessed a high degree of variability. It is generally believed that the emergence of cancer in an individual is the result of a confluence of factors: an inherited genetic weakness, an environmental insult such as radiation or a chemical injury, and the failure of that individual's immune system to respond to the emerging cancer. While significant progress has been made in cancer research, many forms of cancer still remain incurable. For example, pancreatic cancers are among the most lethal of all cancers, because they grow rapidly, metastasize most often before the cancer is discovered, and are resistant to all known modes of therapy. Monoclonal antibodies have been recognized as potentially important agents to use in diagnosing and treating cancer. Monoclonal antibodies react with a single foreign substance (antigen) as do all antibodies, but their most valued characteristics are that they can be selected and created to react with a simple desired antigenic epitope, they can be made in large quantities, and they are relatively innocuous when injected into humans.

### SUMMARY OF THE INVENTION

Preferred embodiments are directed to methods of the treatment of cancer comprising administration of one or more abortogenic agents combined with one or more immunotherapies.

Preferred methods include embodiments wherein said abortogenic agent stimulates immunologically mediated pregnancy resorption/loss.

Preferred methods include embodiments wherein said immunologically mediated pregnancy resorption/loss is associated with immunocyte infiltration into the fetal/placental unit.

Preferred methods include embodiments wherein said immunocyte is a T cell.

Preferred methods include embodiments wherein said T cell is a Th1 cell.

Preferred methods include embodiments wherein said Th1 cell possesses increased production of interferon gamma as compared to IL-4, when stimulated with antibodies to CD3 and CD28.

Preferred methods include embodiments wherein said Th1 cell possesses increased production of interleukin-7 as compared to IL-4, when stimulated with antibodies to CD3 and CD28.

Preferred methods include embodiments wherein said Th1 cell possesses increased production of interleukin-18 as compared to IL-4, when stimulated with antibodies to CD3 and CD28.

Preferred methods include embodiments wherein said Th1 cell possesses increased production of interleukin-27 as compared to IL-4, when stimulated with antibodies to CD3 and CD28.

Preferred methods include embodiments wherein said T cell is a Th17 cell.

Preferred methods include embodiments wherein said Th17 cell produces higher levels of interleukin-17 as compared to IL-4, when stimulated with antibodies to CD3 and CD28.

Preferred methods include embodiments wherein said Th17 cell possesses expression of the RoRgamma transcription factor.

Preferred methods include embodiments wherein said immunocyte is a natural killer cell.

Preferred methods include embodiments wherein said immunocyte is a NKT cell.

Preferred methods include embodiments wherein said immunocyte is a gamma delta T cell.

Preferred methods include embodiments wherein said immunocyte is a monocyte.

Preferred methods include embodiments wherein said immunocyte is a macrophage.

Preferred methods include embodiments wherein said immunocyte is a type 1 macrophage.

Preferred methods include embodiments wherein said immunocyte is a mast cell.

Preferred methods include embodiments wherein said immunocyte is a neutrophil.

Preferred methods include embodiments wherein said immunocyte is a type 1 neutrophil.

Preferred methods include embodiments wherein said immunocyte is a basophil.

Preferred methods include embodiments wherein said immunocyte is a telocyte.

Preferred methods include embodiments wherein said immunocyte is a fibroblast.

Preferred methods include embodiments wherein said immunocyte is a dendritic cell.

Preferred methods include embodiments wherein said dendritic cell is activated.

Preferred methods include embodiments wherein said activated dendritic cell expresses CD1.

Preferred methods include embodiments wherein said activated dendritic cell expresses CD40.

Preferred methods include embodiments wherein said activated dendritic cell expresses CD80.

Preferred methods include embodiments wherein said activated dendritic cell expresses CD86.

Preferred methods include embodiments wherein said activated dendritic cell expresses interleukin-1 receptor.

Preferred methods include embodiments wherein said activated dendritic cell expresses interleukin-4 receptor.

Preferred methods include embodiments wherein said activated dendritic cell expresses GM-CSF receptor.

Preferred methods include embodiments wherein said activated dendritic cell expresses CD1.

Preferred methods include embodiments wherein said immunologically mediated pregnancy resorption/loss is associated with complement activation.

Preferred methods include embodiments wherein said complement activation is caused by stimulation of the alternative pathway.

Preferred methods include embodiments wherein said complement activation is caused by stimulation of the classical pathway.

Preferred methods include embodiments wherein said immunotherapy involves administration of an activator of the innate immune system.

Preferred methods include embodiments wherein said innate immune system activator is a stimulator of a pathogen associated molecular motif receptor.

Preferred methods include embodiments wherein said immunotherapy is administration of an adjuvant together with one or more tumor and/or tumor endothelial antigens.

Preferred methods include embodiments wherein said one or more tumor and/or tumor endothelial antigens are selected from a group comprising of: Antigens known to be found on cancer, and useful for the practice of the invention include: epidermal growth factor receptor (EGFR, EGFR1, ErbB-1, HER1). ErbB-2 (HER2/neu), ErbB-3/HER3, ErbB-4/HER4, EGFR ligand family; insulin-like growth factor receptor (IGFR) family, IGF-binding proteins (IGFBPs), IGFR ligand family (IGF-1R); platelet derived growth factor receptor (PDGFR) family, PDGFR ligand family; fibroblast growth factor receptor (FGFR) family, FGFR ligand family, vascular endothelial growth factor receptor (VEGFR) family, VEGF family; HGF receptor family: TRK receptor family; ephrin (EPH) receptor family: AXL receptor family; leukocyte tyrosine kinase (LTK) receptor family; TIE receptor family, angiopoietin 1, 2; receptor tyrosine kinase-like orphan receptor (ROR) receptor family; discoidin domain receptor (DDR) family; RET receptor family; KLG receptor family; RYK receptor family; MuSK receptor family; Transforming growth factor alpha (TGF-.alpha.), TGF-.alpha. receptor; Transforming growth factor-beta (TGF-.beta.), TGF-.beta. receptor; Interleukin .beta. receptor alpha2 chain (IL13Ralpha2), Interleukin-6 (IL-6), 1L-6 receptor, interleukin-4, IL-4 receptor, Cytokine receptors, Class I (hematopoietin family) and Class II (interferon/1L-10 family) receptors, tumor necrosis factor (TNF) family, TNF-.alpha., tumor necrosis factor (TNF) receptor superfamily (TNTRSF), death receptor family, TRAIL-receptor; cancer-testis (CT) antigens, lineage-specific antigens, differentiation antigens, alpha-actinin-4, ARTC1, breakpoint cluster region-Abelson (Bcr-abl) fusion products, B-RAF, caspase-5 (CASP-5), caspase-8 (CASP-8), beta-catenin (CTNNB1), cell division cycle 27 (CDC27), cyclin-dependent kinase 4 (CDK4), CDKN2A, COA-1, dek-can fusion protein, EFTUD-2, Elongation factor 2 (ELF2), Ets variant gene 6/acute myeloid leukemia 1 gene ETS (ETC6-AML1) fusion protein, fibronectin (FN), GPNMB, low density lipid receptor/GDP-L fucose: beta-Dgalactose 2-alpha-Lfucosyltraosferase (LDLR/FUT) fusion protein, HLA-A2, MLA-Al1, heat shock protein 70-2 mutated (HSP70-2M), KIAA0205, MART2, melanoma ubiquitous mutated 1, 2, 3 (MUM-1, 2, 3), prostatic acid phosphatase (PAP), neo-PAP, Myosin class 1, NFYC, OGT, OS-9, pml-RARalpha fusion protein, PRDX5, PTPRK, K-ras (KRAS2), N-ras (NRAS), HRAS, RBAF600, SIRT12, SNRPD1, SYT-SSX1 or-SSX2 fusion protein, Triosephosphate Isomerase, BAGE, BAGE-1, BAGE-2, 3, 4, 5, GAGE-1, 2, 3, 4, 5, 6, 7, 8, GnT-V (aberrant N-acetyl glucosaminyl transferase V, MGAT5), HERV-K MEL, KK-LC, KM-HN-1, LAGE, LAGE-1, CTL-recognized antigen on melanoma (CAMEL), MAGE-A1 (MAGE-1). MAGE-A2, MAGE-A3, MAGE-A4, MAGE-AS, MAGE-A6, MAGE-A8, MAGE-A9, MAGE-A10. MAGE-A11, MAGE-A12, MAGE-3, MAGE-B1, MAGE-B2, MAGE-B5. MAGE-B6, MAGE-C1, MAGE-C2, mucin 1 (MUC1), MART-1/Melan-A (MLANA), gp100, gp100/Pme117 (S1LV), tyrosinase (TYR), TRP-1, HAGE, NA-88, NY-ESO-1, NY-ESO-1/LAGE-2, SAGE, Sp17. SSX-1, 2, 3, 4, TRP2-1NT2, carcino-embryonic antigen (CEA), Kallikrein 4, mammaglobin-A, OA1, prostate specific antigen (PSA), prostate specific membrane antigen, TRP-1/, 75. TRP-2 adipophilin, interferon inducible protein absent in melanoma 2 (AIM-2). BING-4, CPSF, cyclin D1, epithelial cell adhesion molecule (Ep-CAM), EpbA3, fibroblast growth factor-5 (FGF-5), glycoprotein 250 (gp250intestinal carboxyl esterase (iCE), alpha-feto protein (AFP), M-CSF, mdm-2, MUCI, p53 (TP53), PBF, PRAME, PSMA, RAGE-1, RNF43, RU2AS, SOX10, STEAP1, survivin (BIRCS), human telomerase reverse transcriptase (hTERT), telomerase, Wilms' tumor gene (WT1), SYCP1, BRDT, SPANX, XAGE, ADAM2, PAGE-5, LIP1, CTAGE-1, CSAGE, MMA1, CAGE, BORIS, HOM-TES-85, AF15q14, HCA66I, LDHC, MORC, SGY-1, SPO11, TPX1, NY-SAR-35, FTHLI7, NXF2 TDRD1, TEX 15, FATE, TPTE, immunoglobulin idiotypes, Bence-Jones protein, estrogen receptors (ER), androgen receptors (AR), CD40, CD30, CD20, CD19, CD33, CD4, CD25, CD3, cancer antigen 72-4 (CA 72-4), cancer antigen 15-3 (CA 15-3), cancer antigen 27-29 (CA 27-29), cancer antigen 125 (CA 125), cancer antigen 19-9 (CA 19-9), beta-human chorionic gonadotropin, 1-2 microglobulin, squamous cell carcinoma antigen, neuron-specific enolase, heat shock protein gp96. GM2, sargramostim, CTLA-4, 707 alanine proline (707-AP), adenocarcinoma antigen recognized by T cells 4 (ART-4), carcinoembryogenic antigen peptide-1 (CAP-1), calcium-activated chloride channel-2 (CLCA2), cyclophilin B (Cyp-B), and human signet ring tumor-2 (HST-2).

Preferred methods include embodiments wherein said abortogenic agent is an inhibitor of the progesterone signaling pathway.

Preferred methods include embodiments wherein said abortogenic agent is an inhibitor of the glucocorticoid signaling pathway.

Preferred methods include embodiments wherein said abortogenic agent is an inhibitor of the glucocorticoid and progesterone signaling pathway.

Preferred methods include embodiments wherein said abortogenic agent is RU-486 or an analogue thereof.

Preferred methods include embodiments wherein said abortogenic agent is an inhibitor of indolamine 2,3 dioxygenase.

Preferred methods include embodiments wherein said inhibitor of 2,3 dioxygenase is 2-MT.

Preferred methods include embodiments wherein said inhibitor of 2,3 dioxygenase is quadramune.

Preferred methods include embodiments wherein said inhibitor of 2,3 dioxygenase is 2-MT.

Preferred methods include embodiments wherein said immunotherapy is an anti-angiogenic immunotherapy.

Preferred methods include embodiments wherein said anti-angiogenic immunotherapy is endothelial cell based.

Preferred methods include embodiments wherein said endothelial based immunotherapy is ValloVax.

Preferred methods include embodiments in which augmenting an immune response in a mammal through the administration of mifepristone or a derivative thereof is accomplished by providing it at an at an immunologically effective concentration.

Preferred methods include embodiments wherein said mifepristone derivative is an agent capable of antagonizing function of glucocorticoid receptors.

Preferred methods include embodiments wherein said mifepristone derivative is an agent capable of suppressing the hypothalamic pituitary adrenal axis (HPA).

Preferred methods include embodiments wherein said immunologically effective dose is equivalent to a parenteral dose in a human immunologically similar to a dose of 25 ug/g intraperitoneal injection into a 6-8 week C57/B6 mouse administered on a daily basis.

Preferred methods include embodiments wherein said immunologically effective dose is the dose needed to achieve a reduction in T regulatory cell numbers or activity in a patient.

Preferred methods include embodiments wherein said T regulatory cells are a cell population capable of inhibiting an immune response through contact dependent or independent mechanisms.

Preferred methods include embodiments wherein said immunologically effective dose is the dose needed to achieve a reduction in immune suppressive cytokines.

Preferred methods include embodiments wherein said immune suppressive cytokines are selected from a group comprising of: a) IL-4; b) IL-6; c) IL-10; d) IL-13; e) IL-20; f) TGF-beta; and g) LIF.

Preferred methods include embodiments wherein said immunologically effective dose is the dose needed to achieve reduction of an immune suppressive factor.

Preferred methods include embodiments wherein said immune suppressive factor is systemically acting.

Preferred methods include embodiments wherein said systemically acting immune suppressive factor is selected from a group comprising of: a) soluble HLA-G; b) soluble MICA/B; c) PIGF; d) fas ligand (FasL) expressing exosomes; e) interleukin 1 receptor antagonist; f) soluble FasL; and g) soluble MHC.

Preferred methods include embodiments wherein said immune suppressive factor is membrane bound.

Preferred methods include embodiments wherein said membrane/cell bound immune suppressive factor is selected from a group comprising of: a) FasL; b) PD-1 ligand; c) PD-2 ligand; d) indolamine 2,3 deoxygenase;and e) arginase.

### BRIEF DESCRIPTION OF DRAWINGS

FIG. 1 is a bar graph showing the results of administering VALLOVAX™ and/or RU-486 in cancer modeled mice.

### DETAILED DESCRIPTION OF THE INVENTION

At least one specification heading is required. Please delete this heading section if it is not applicable to your application. For more information regarding the headings of the specification, please see MPEP 608.01(a).

The invention discloses a novel way of stimulating immune responses to cancer endothelial cells through administration of abortogenic agents together with immunotherapeutic agents.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

The term “about” is used to indicate that a value includes the inherent variation of error for the device, the method being employed to determine the value, or the variation that exists among the study subjects.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. Other specific types of cancer include cinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrmcous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti, The term “sarcoma” generally refers to a tumor which is made up of a substance like the embryonic connective tissue and is generally composed of closely packed cells embedded in a fibrillar, heterogeneous, or homogeneous substance. Sarcomas include, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma. Additional exemplary neoplasias include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

In some particular embodiments of the invention, the cancer treated is a melanoma. The term “melanoma” is taken to mean a tumor arising from the melanocytic system of the skin and other organs. Melanomas include, for example, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma.

The term “chimeric antigen receptors (CARs),” as used herein, may refer to artificial T-cell receptors, chimeric T-cell receptors, or chimeric immunoreceptors, for example, and encompass engineered receptors that graft an artificial specificity onto a particular immune effector cell. CARs may be employed to impart the specificity of a monoclonal antibody onto a T cell, thereby allowing a large number of specific T cells to be generated, for example, for use in adoptive cell therapy. In specific embodiments, CARs direct specificity of the cell to a tumor associated antigen, for example. In some embodiments, CARs comprise an intracellular activation domain, a transmembrane domain, and an extracellular domain comprising a tumor associated antigen binding region. In particular aspects, CARs comprise fusions of single-chain variable fragments (scFv) derived from monoclonal antibodies, fused to CD3-zeta a transmembrane domain and endodomain. The specificity of other CAR designs may be derived from ligands of receptors (e.g., peptides) or from pattern-recognition receptors, such as Dectins. In certain cases, the spacing of the antigen-recognition domain can be modified to reduce activation-induced cell death. In certain cases, CARs comprise domains for additional co-stimulatory signaling, such as CD3.zeta., FcR, CD27, CD28, CD137, DAP10, DAP12 and/or OX40. In some cases, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

The term “Costimulatory ligand” or “costimulatory molecule” as used herein, includes a molecule on an antigen presenting cell that specifically binds a cognate co-stimulatory molecule on a T cell. Binding of the costimulatory ligand provides a signal that mediates a T cell response, including, but not limited to, proliferation, activation, differentiation, and the like. A costimulatory ligand induces a signal that is in addition to the primary signal provided by a stimulatory molecule, for instance, by binding of a T cell receptor (TCR)/CD3 complex with a major histocompatibility complex (MHC) molecule loaded with peptide. A co-stimulatory ligand can include, but is not limited to, CD7, B7-1 (CD80), B7-2 (CD86), programmed death (PD) L1, PD-L2, 4-1BB ligand, OX40 ligand, inducible costimulatory ligand (ICOS-L), intercellular adhesion molecule (ICAM), CD30 ligand, CD40, CD70, CD83, human leukocyte antigen G (HLA-G), MHC class I chain-related protein A (MICA), MHC class I chain-related protein B (MICB), herpes virus entry mediator (HVEM), lymphotoxin beta receptor, 3/TR6, immunoglobulin-like transcript (ILT) 3, ILT4, an agonist or antibody that binds Toll ligand receptor and a ligand that specifically binds with B7-H3. A co-stimulatory ligand includes, without limitation, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, tumor necrosis factor superfamily member 14 (TNFSF14 or LIGHT), natural killer cell receptor C (NKG2C), B7-H3, and a ligand that specifically binds with CD83.

The term “cytokine”, as used herein, refers to a non-antibody protein that is released by one cell in response to contact with a specific antigen, wherein the cytokine interacts with a second cell to mediate a response in the second cell. A cytokine can be endogenously expressed by a cell or administered to a subject. Cytokines may be released by immune cells, including macrophages, B cells, T cells, and mast cells to propagate an immune response. Cytokines can induce various responses in the recipient cell. Cytokines can include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines can promote an inflammatory response.

Examples of homeostatic cytokines include, but are not limited to, IL-2, IL-4, IL-5, IL-7, IL-10, IL-12p40, IL-12p70, IL-15, and interferon (IFN) gamma. Examples of pro-inflammatory cytokines include, but are not limited to, IL-1a, IL-1b, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

The term “exogenous,” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, the term refers to a cell that was isolated and subsequently introduced to other cells or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from where it would be in natural cells, or is otherwise flanked by a different nucleic acid sequence than that found in nature.

The term “expression construct” or “expression cassette” refers to a nucleic acid molecule that is capable of directing transcription. An expression construct includes, at a minimum, one or more transcriptional control elements (such as promoters, enhancers or a structure functionally equivalent thereof) that direct gene expression in one or more desired cell types, tissues or organs. Additional elements, such as a transcription termination signal, may also be included.

The term “vector” or “construct” (sometimes referred to as a gene delivery system or gene transfer “vehicle”) refers to a macromolecule or complex of molecules comprising a polynucleotide to be delivered to a host cell, either in vitro or in vivo.

The term “plasmid,” a common type of a vector, is an extra-chromosomal DNA molecule separate from the chromosomal DNA that is capable of replicating independently of the chromosomal DNA. In certain cases, it is circular and double-stranded.

The term “origin of replication” (“ori”) or “replication origin” is a DNA sequence, e.g., in a lymphotrophic herpes virus, that when present in a plasmid in a cell is capable of maintaining linked sequences in the plasmid and/or a site at or near where DNA synthesis initiates. As an example, an ori for EBV (Ebstein-Barr virus) includes FR sequences (20 imperfect copies of a 30 bp repeat), and preferably DS sequences; however, other sites in EBV bind EBNA-1, e.g., Rep* sequences can substitute for DS as an origin of replication (Kirshmaier and Sugden, 1998). Thus, a replication origin of EBV includes FR, DS or Rep* sequences or any functionally equivalent sequences through nucleic acid modifications or synthetic combination derived therefrom. For example, methods of the present disclosure may also use genetically engineered replication origin of EBV, such as by insertion or mutation of individual elements.

The term “gene,” “polynucleotide,” “coding region,” “sequence,” “segment,” “fragment,” or “transgene” that “encodes” a particular protein, is a nucleic acid molecule that is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “control elements” refers collectively to promoter regions, polyadenylation signals, transcription termination sequences, upstream regulatory domains, origins of replication, internal ribosome entry sites (IRES), enhancers, splice junctions, and the like, which collectively provide for the replication, transcription, post-transcriptional processing, and translation of a coding sequence in a recipient cell. Not all of these control elements need be present so long as the selected coding sequence is capable of being replicated, transcribed, and translated in an appropriate host cell.

The term “promoter” is used herein in its ordinary sense to refer to a nucleotide region comprising a DNA regulatory sequence, wherein the regulatory sequence is derived from a gene that is capable of binding RNA polymerase and initiating transcription of a downstream (3′ direction) coding sequence. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

The term “enhancer” is meant a nucleic acid sequence that, when positioned proximate to a promoter, confers increased transcription activity relative to the transcription activity resulting from the promoter in the absence of the enhancer domain.

The term “operably linked” or co-expressed” with reference to nucleic acid molecules is meant that two or more nucleic acid molecules (e.g., a nucleic acid molecule to be transcribed, a promoter, and an enhancer element) are connected in such a way as to permit transcription of the nucleic acid molecule. “Operably linked” or “co-expressed” with reference to peptide and/or polypeptide molecules means that two or more peptide and/or polypeptide molecules are connected in such a way as to yield a single polypeptide chain, i.e., a fusion polypeptide, having at least one property of each peptide and/or polypeptide component of the fusion. The fusion polypeptide is preferably chimeric, i.e., composed of heterologous molecules.

The term “Homology” refers to the percent of identity between two polynucleotides or two polypeptides. The correspondence between one sequence and another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions that promote the formation of stable duplexes between homologous regions, followed by digestion with single strand-specific nuclease(s), and size determination of the digested fragments. Two DNA, or two polypeptide, sequences are “substantially homologous” to each other when at least about 80%, preferably at least about 90%, and most preferably at least about 95% of the nucleotides, or amino acids, respectively match over a defined length of the molecules, as determined using the methods above.

The term “cell” is herein used in its broadest sense in the art and refers to a living body that is a structural unit of tissue of a multicellular organism, is surrounded by a membrane structure that isolates it from the outside, has the capability of self-replicating, and has genetic information and a mechanism for expressing it. Cells used herein may be naturally-occurring cells or artificially modified cells (e.g., fusion cells, genetically modified cells, etc.).

The current invention is based on the unexpected finding of augment antitumor activity through the combination of an immune stimulant suppressing angiogenesis together with mifepristone. Augmentation of antitumor and/or antiangiongenic activity of an immune stimulant in the context of cancer has many practical implications. Without being bound to theory, the reduction of tumor growth may be associated with derepression of glucocorticoid-mediated immune suppression. Accordingly, one embodiment of the current invention is the use of other immune stimulants together with mifepristone. Specifically, tumor vaccination has been associated with numerous clinical failures. One cause of these failures may be the endogenous activation of the HPA and associated rise in immune suppressive glucocorticoids. Immune suppression by cancer has been well-documented in advanced cancer patients possessing a variety of malignancies. These include pancreatic cancer, breast cancer, renal cancer, colorectal cancer and melanoma. Suppression is noted by diminished T cell proliferative response, diminished ability to produce IFN-g, and diminished ability to induce recall responses to normal antigen. Immune suppression does not allow for proper eradication of tumors by immunotherapy, or by the body's natural mechanisms. Correlation between immune suppression and poor prognosis has been extensively notes. There are several mechanisms by which tumor immune suppression occurs. One, which is concurrent with activation of the HPA axis and associated oxidative stress is the loss of the signaling molecule TCR-zeta. Tumor cells induce cleavage of the T cell receptor zeta (TCR-z) chain through a caspase-3 dependent manner. This is both FasL-dependent and independent. Since TCR-zeta is critical for signal transduction, the T cells become unable to respond to tumor antigens. Originally, the suppressed level of TCR-zeta was described in tumor bearing mice and subsequently in patients.

It appears that several of the immune suppressive mechanisms used by the tumor are all downstream of HPA activation, or as a chronic inflammation. The ability of mifepristone to decrease said chronic inflammation is one of the embodiments of the current invention. In this context, we will explain how chronic inflammation is associated with cancer immune suppression. One of the causes of this cancer associated chronic inflammation is the tumor infiltrating macrophages. Tumors usually associated with macrophage infiltration, this is correlated with tumor stage and is believed to contribute to tumor progression by stimulation of angiogenesis.

In addition to oxidative stress elaborated by tumor associated macrophages, the presence of the tumor itself causes systemic changes associated with chronic inflammation.

Erythrocyte sedimentation ration, C-reactive protein and IL-6 are markers of inflammatory stress used to designate progression of diseases such as arthritis. Interestingly advanced cancer patients possess all of these inflammatory markers. Another marker of chronic inflammation is decreased albumin synthesis by the liver, this is also seen in cancer patients and is believed to contribute, in part, to cachexia. In addition, the inflammatory marker fibrinogen D-dimers is also higher in cancer patients as opposed to controls.

VALLOVAX™ is a trade name for a placentally and endothelial derived antiangiogenic immunotherapeutic which has been demonstrated to suppress cancer independent of tissue of origin. More specifically, VALLOVAX™ comprises placental derived endothelial cells treated with IFN-gamma. VALLOVAX™ is made as follows: Full term human placentas are collected from delivery room under informed consent. Fetal membranes are manually peeled back and the villous tissue is isolated from the placental structure. Villous tissue is subsequently washed with cold saline to remove blood and scissors are used to mechanically digest the tissue. Lots of 25 grams of minced tissue are incubated with approximately 50 ml of HBSS with 25 mM of HEPES and 0.28% collagenase, 0.25% dispase, and 0.01% DNAse at 37 Celsius.

The mixture of minced placental villus tissue and digesting solution is incubated under stirring conditions for three incubation periods of 20 minutes each. Ten minutes after the first incubation period and immediately after the second and third incubation periods, the DNAse was added to make up a total concentration of DNase, by volume, of 0.01%. In the first and second incubations, the incubation flask is set at an angle, and the tissue fragments allowed to settle for approximately 1 minute, with 35 ml of the supernatant cell suspension being collected and replaced by 38 ml (after the first digestion) or 28 ml (after the second digestion) of fresh digestion solution. After the third digestion the whole supernatant is collected. The supernatant collected from all three incubations is then pooled and is poured through approximately four layers of sterile gauze and through one layer of 70 micrometer polyester mesh. The filtered solution is then centrifuged for 1000 g for 10 minutes through diluted new born calf serum, said new born calf serum diluted at a ratio of 1 volume saline to 7 volumes of new born calf serum.

The pooled pellet is then resuspended in 35 ml of warm DMEM with 25 mM HEPES containing 5 mg DNase I. The suspension is subsequently mixed with 10 ml of 90% Percoll to give a final density of 1.027 g/ml and centrifuged at 550 g for 10 minutes with the centrifuge brake off. The pellet is then washed in HBSS and cells incubated for 48 hours in complete DMEM media. After 3-4 passages cells are incubating in media containing 100 IU of IFN-gamma per ml.

RU-486 is an FDA cleared abortion inducing agent that functions through inhibition of progesterone and glucocorticoid signaling, thus blocking production of immune suppressive pathways. In the current study we show that RU-486 enhances therapeutic activity of ValloVax in the triple negative breast cancer 4T1 model. Administration of RU-486 decreased breast cancer induced myeloid suppressor cells, increased T cell reactivity in vitro, and enhance generation of CD8 cells which were capable of adoptively transferring immunity. These data support the generation of combining agents.

### EXAMPLE

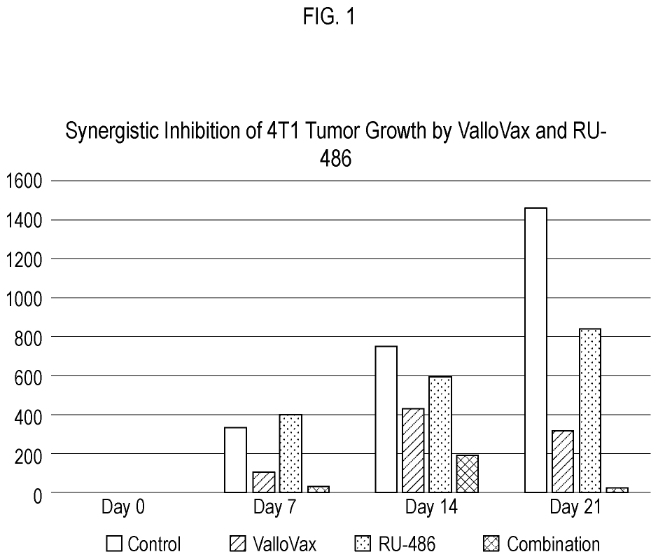

Female BALB/c mice were administered 4T1 cells at a concentration of 1 million cells per mouse. VALLOVAX™ at a concentration of 500,000 cells per mouse was administered alone, RU-486 was administered alone (25 μg/g dose) and in combination of RU-486 and VALLOVAX™. Tumor growth was significantly inhibited by ValloVax and RU-486 combination. Results are shown in FIG. 1

1. A method of the treatment of cancer comprising administration of one or more abortogenic agents combined with one or more immunotherapies.

2. The method of claim 1, wherein said abortogenic agent stimulates immunologically mediated pregnancy resorption/loss.

3. The method of claim 2, wherein said immunologically mediated pregnancy resorption/loss is associated with immunocyte infiltration into the fetal/placental unit.

4. The method of claim 1, wherein said immunotherapy is administration of an adjuvant together with one or more tumor and/or tumor endothelial antigens.

5. The method of claim 4, wherein said one or more tumor and/or tumor endothelial antigens are selected from the group consisting of: epidermal growth factor receptor (EGFR, EGFR1, ErbB-1, HER1). ErbB-2 (HER2/neu), ErbB-3/HER3, ErbB-4/HER4, EGFR ligand family; insulin-like growth factor receptor (IGFR) family, IGF-binding proteins (IGFBPs), IGFR ligand family (IGF-1R);
platelet derived growth factor receptor (PDGFR) family, PDGFR ligand family; fibroblast growth factor receptor (FGFR) family, FGFR ligand family, vascular endothelial growth factor receptor (VEGFR) family, VEGF family; HGF receptor family: TRK receptor family; ephrin (EPH) receptor family: AXL receptor family; leukocyte tyrosine kinase (LTK) receptor family; TIE receptor family, angiopoietin 1, 2; receptor tyrosine kinase-like orphan receptor (ROR) receptor family; discoidin domain receptor (DDR) family; RET receptor family; KLG receptor family; RYK receptor family;
MuSK receptor family; Transforming growth factor alpha (TGF-.alpha.), TGF-.alpha. receptor;
Transforming growth factor-beta (TGF-.beta.), TGF-.beta. receptor; Interleukin .beta. receptor alpha2 chain (IL13Ralpha2), Interleukin-6 (IL-6), 1L-6 receptor, interleukin-4, IL-4 receptor, Cytokine receptors, Class I (hematopoietin family) and Class II (interferon/1L-10 family) receptors, tumor necrosis factor (TNF) family, TNF-.alpha., tumor necrosis factor (TNF) receptor superfamily (TNTRSF), death receptor family, TRAIL-receptor; cancer-testis (CT) antigens, lineage-specific antigens, differentiation antigens, alpha-actinin-4, ARTC1, breakpoint cluster region-Abelson (Bcr-abl) fusion products, B-RAF, caspase-5 (CASP-5), caspase-8 (CASP-8), beta-catenin (CTNNB1), cell division cycle 27 (CDC27), cyclin-dependent kinase 4 (CDK4), CDKN2A, COA-1, dek-can fusion protein, EFTUD-2, Elongation factor 2 (ELF2), Ets variant gene 6/acute myeloid leukemia 1 gene ETS (ETC6-AML1) fusion protein, fibronectin (FN), GPNMB, low density lipid receptor/GDP-L fucose: beta-Dgalactose 2-alpha-Lfucosyltraosferase (LDLR/FUT) fusion protein, HLA-A2, MLA-Al1, heat shock protein 70-2 mutated (HSP70-2M), KIAA0205, MART2, melanoma ubiquitous mutated 1, 2, 3 (MUM-1, 2, 3), prostatic acid phosphatase (PAP), neo-PAP, Myosin class 1, NFYC, OGT, OS-9, pml-RARalpha fusion protein, PRDX5, PTPRK, K-ras (KRAS2), N-ras (NRAS), HRAS, RBAF600, SIRT12, SNRPD1, SYT-SSX1 or-SSX2 fusion protein, Triosephosphate Isomerase, BAGE, BAGE-1, BAGE-2, 3, 4, 5, GAGE-1, 2, 3, 4, 5, 6, 7, 8, GnT-V (aberrant N-acetyl glucosaminyl transferase V, MGAT5), HERV-K MEL, KK-LC, KM-HN-1, LAGE, LAGE-1, CTL-recognized antigen on melanoma (CAMEL), MAGE-A1 (MAGE-1). MAGE-A2, MAGE-A3, MAGE-A4, MAGE-AS, MAGE-A6, MAGE-A8, MAGE-A9, MAGE-A10. MAGE-A11, MAGE-A12, MAGE-3, MAGE-B1, MAGE-B2, MAGE-B5. MAGE-B6, MAGE-C1, MAGE-C2, mucin 1 (MUC1), MART-1/Melan-A (MLANA), gp100, gp100/Pme117 (S1LV), tyrosinase (TYR), TRP-1, HAGE, NA-88, NY-ESO-1, NY-ESO-1/LAGE-2, SAGE, Sp17. SSX-1, 2, 3, 4, TRP2-1NT2, carcino-embryonic antigen (CEA), Kallikrein 4, mammaglobin-A, OA1, prostate specific antigen (PSA), prostate specific membrane antigen, TRP-1/, 75. TRP-2 adipophilin, interferon inducible protein absent in melanoma 2 (AIM-2). BING-4, CPSF, cyclin D1, epithelial cell adhesion molecule (Ep-CAM), EpbA3, fibroblast growth factor-5 (FGF-5), glycoprotein 250 (gp250intestinal carboxyl esterase (iCE), alpha-feto protein (AFP), M-CSF, mdm-2, MUCI, p53 (TP53), PBF, PRAME, PSMA, RAGE-1, RNF43, RU2AS, SOX10, STEAP1, survivin (BIRCS), human telomerase reverse transcriptase (hTERT), telomerase, Wilms' tumor gene (WT1), SYCP1, BRDT, SPANX, XAGE, ADAM2, PAGE-5, LIP1, CTAGE-1, CSAGE, MMA1, CAGE, BORIS, HOM-TES-85, AF15q14, HCA66I, LDHC, MORC, SGY-1, SPO11, TPX1, NY-SAR-35, FTHLI7, NXF2 TDRD1, TEX 15, FATE, TPTE, immunoglobulin idiotypes, Bence-Jones protein, estrogen receptors (ER), androgen receptors (AR), CD40, CD30, CD20, CD19, CD33, CD4, CD25, CD3, cancer antigen 72-4 (CA 72-4), cancer antigen 15-3 (CA 15-3), cancer antigen 27-29 (CA 27-29), cancer antigen 125 (CA 125), cancer antigen 19-9 (CA 19-9), beta-human chorionic gonadotropin, 1-2 microglobulin, squamous cell carcinoma antigen, neuron-specific enolase, heat shock protein gp96. GM2, sargramostim, CTLA-4, 707 alanine proline (707-AP), adenocarcinoma antigen recognized by T cells 4 (ART-4), carcinoembryogenic antigen peptide-1 (CAP-1), calcium-activated chloride channel-2 (CLCA2), cyclophilin B (Cyp-B), and human signet ring tumor-2 (HST-2).

6. The method of claim 1, wherein said abortogenic agent is an inhibitor of the progesterone signaling pathway.

7. The method of claim 1, wherein said abortogenic agent is an inhibitor of the glucocorticoid signaling pathway.

8. The method of claim 1, wherein said abortogenic agent is an inhibitor of the glucocorticoid and progesterone signaling pathway.

9. The method of claim 1, wherein said abortogenic agent is mifepristone or an analogue thereof.

10. The method of claim 1, wherein said abortogenic agent is an inhibitor of indolamine 2,3 dioxygenase.

11. The method of claim 10, wherein said inhibitor of 2,3 dioxygenase is 2-MT.

12. The method of claim 10, wherein said inhibitor of 2,3 dioxygenase is quadramune.

13. The method of claim 10, wherein said inhibitor of 2,3 dioxygenase is 2-MT.

14. The method of claim 1, wherein said immunotherapy is an anti-angiogenic immunotherapy.

15. The method of claim 14, wherein said anti-angiogenic immunotherapy is endothelial cell based.

16. The method of claim 15, wherein said endothelial based immunotherapy comprises placental derived endothelial progenitor cells pretreated with interferon gamma.

17. The method of claim 9, wherein said mifepristone is administered at a dose of 100-400 mg/day.
